(19) United States
(12) Patent Application Publication
(10) Pub. No.: US 2003/0075185 A1
(43) Pub. Date: Apr. 24, 2003

(54) Extruded earplugs with printed indicia

(76) Inventor: Volker R. Ulbrich, Worcester, MA (US)

Correspondence Address: Richard L. Sampson, Sampson & Associates, 50 Congress Street, Boston, MA, 02109, US

(21) Appl. No.: 09/981,854

(22) Filed: Oct. 18, 2001

(51) Int. Cl. A61F 11/00

(52) U.S. Cl. 128/864

(57) ABSTRACT

A monolithic earplug is provided, which is compressible or deformable. Individual earplugs are cut from an extruded elastomeric thermoplastic material, providing for a relatively rapid and efficient fabrication method. Decorative and/or promotional markings may be disposed on a surface thereof either before or after cutting.

### BACKGROUND OF THE INVENTION

(1) Field of the Invention

The present invention relates generally to earplugs and more particularly to extruded earplugs for attenuating sound having decorative and/or promotional markings thereon and a method for fabrication thereof.

(2) Background Information

Earplugs configured for insertion into a person's ear canals to dampen sound and/or prevent the entry of foreign matter are well known. Historically, these earplugs were manufactured from fibrous materials such as cotton. In more recent times, earplugs have been produced from polymer-based materials, which tend to be more durable than the fibrous materials formerly used. Monolithic, polymer-based earplugs have been typically (and conventionally) fabricated by one of two methods; die cutting plugs from sheets of cellular materials, such as vinyl, or molding a polymer-based material into a desired shape (see for example U.S. Pat. Nos. 3,872,559 and 4,774,938 to Leight). In an attempt to provide earplugs having sufficient longitudinal rigidity to allow easy insertion to the ear canals and simultaneously sufficient compressibility to allow snug yet comfortable wearer fit, multi-component earplugs having a rigid core surrounded by a more compressive sheath have also been taught (see for example U.S. Pat. No. 4,434,794 to Leight and U.S. Pat. No. 5,188,123 to Gardner). Further, in an attempt to overcome the manufacturing complexities associated with molded construction, Williams, in U.S. Pat. No. 5,573,015, discloses a multi-component earplug fabricated using an extrusion process.

More recently, Smith et al., in commonly assigned U.S. patent application Ser. No. 09/670,678, which is fully incorporated herein by reference and hereinbelow referred to as the '678 application, disclose an extruded monolithic foam earplug. The earplug disclosed in the '678 application provides several advantages, including ease and efficiency of manufacturing, user comfort and ease of use, and greater sound attenuation.

Nevertheless, notwithstanding the substantial improvements made by Smith et al., there exists a need for an improved earplug and method for fabrication thereof. In particular there exists a need for an earplug including the properties and advantages disclosed in the '678 application, but including further improvements.

### SUMMARY OF THE INVENTION

One aspect of the present invention includes a method for fabricating an earplug. The method includes extruding a monolithic body of foamed elastomeric thermoplastic material about 5 to 20 millimeters in transverse dimension, at least partially cutting the body into discrete pieces about 10-35 millimeters in length to form individual earplugs, and disposing markings on a surface of the earplug. In one variation of this aspect the markings are disposed on the surface of the earplug using an ink jet (or bubble jet) print head, a stamp, an offset printing press, a dye extruder, a screen printer, or combinations thereof.

In another aspect, this invention includes an earplug sized and shaped for being received in a human ear canal. The earplug includes an extruded monolithic body of foamed elastomeric thermoplastic material about 5 to 20 millimeters in transverse dimension having a length of about 10-35 millimeters and markings disposed on at least one surface of the earplug. In one variation of this aspect the markings include decorative artwork, promotional markings, brand names, logos, indicia, trademarks, advertising, or combinations thereof.

In yet another aspect, this invention includes a monolithic earplug formed by the process of disposing a PVC-based material within an extruder under heat and pressure, incorporating a blowing agent into the material, extruding the material in a longitudinal direction from a die into an ambient environment in which the blowing agent foams the extrudate, the extrudate having a transverse cross-sectional dimension of about 5 to 20 millimeters, cutting the extrudate at a substantially 90 degree angle to the longitudinal direction as the extrudate emerges from the die and prior to substantially complete cooling and expansion thereof, wherein a convex, skinned surface is formed at the cut ends as the extrudate expands and cools to form a monolithic earplug having a density of about 6 to 12 pcf (96 to 192 kg/m³), and a rate of recovery from 80 percent compression sufficient to recover about 90 percent or less of its initial volume in 45 seconds, and after being compressed under a 5 pound weight for 6 seconds, to recover about 90 percent or more of its initial volume in 90 seconds, and disposing markings on at least one surface of the earplugs.

### BRIEF DESCRIPTION OF THE DRAWINGS

FIG. 1 is a sectional view of an earplug according to the present invention inserted in a wearer's ear;

FIG. 2 is a perspective view of an earplug according to an embodiment of the present invention having squared-off, planar end portions;

FIG. 3 is a cross-sectional view of the earplug shown in FIGS. 2 and 4, as shown along line 3-3 of FIG. 4;

FIG. 4 is a longitudinal cross-sectional view taken along line 4-4 of FIG. 3;

FIG. 5 is a perspective view of an alternate embodiment of the present invention having convex end portions;

FIG. 6 is a longitudinal cross-sectional view of the earplug shown in FIG. 5;

FIG. 7 is a perspective view of an earplug according to an additional embodiment of the present invention having rounded shaped end portions;

FIG. 8 is a perspective view of an earplug according to yet an additional embodiment of the present invention including markings on a surface thereof;

FIG. 9 is a view of one end of the earplug of FIG. 8;

FIG. 10 is a perspective view of an earplug according to still an additional embodiment of the present invention including markings on a surface thereof;

FIG. 11 is a schematic representation of a production apparatus for the earplugs in accordance with the present invention;

FIG. 12 is a schematic representation of another production apparatus for the earplugs in accordance with the present invention;

FIG. 13 is a schematic representation of yet another production apparatus for the earplugs in accordance with the present invention;

FIG. 14 is a schematic representation of still another production apparatus for the earplugs in accordance with the present invention; and

FIG. 15 is a graphical comparison of sound attenuation test results of the earplug of this invention relative to earplugs of the prior art.

### DETAILED DESCRIPTION

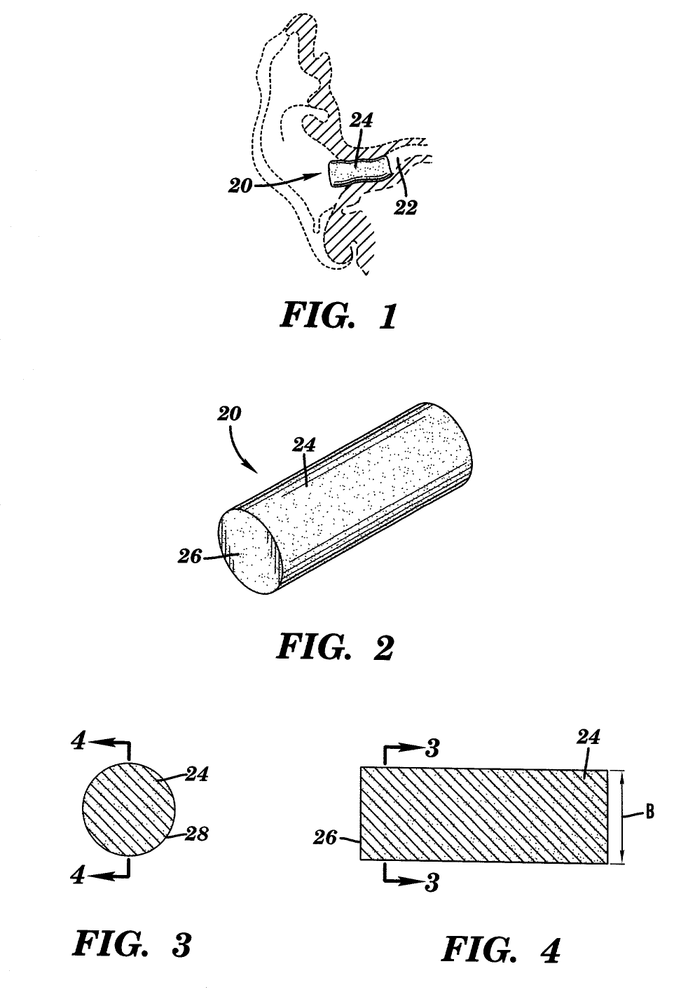

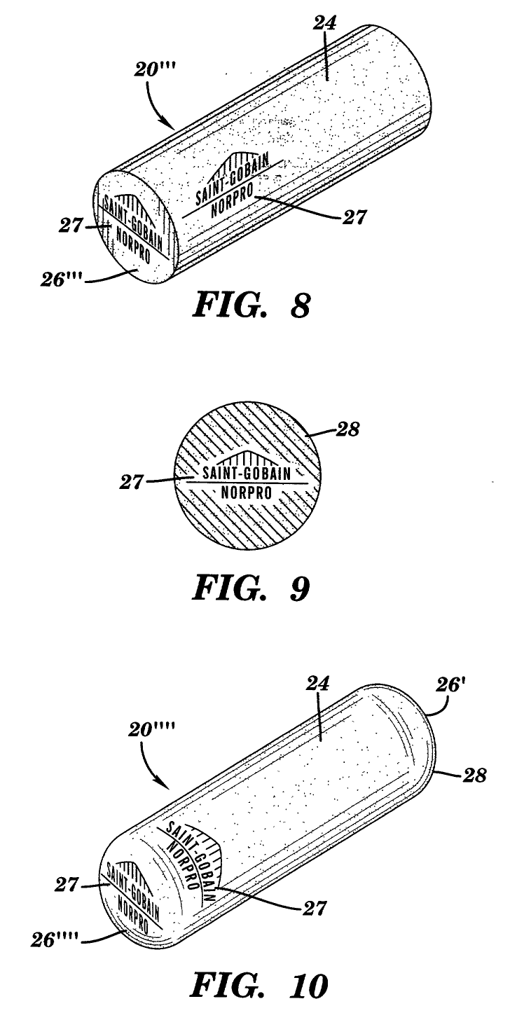

Referring to FIG. 1, the present invention is directed to an earplug 20 configured for insertion in a human ear canal 22 for attenuating sound and protecting against entry of foreign matter into the ear canal 22. The earplug 20 of the present invention includes a monolithic body 24. The body 24 is sufficiently soft and flexible to provide a comfortable fit for the wearer. The earplug 20 is desirably compressible or deformable in order that it can be compressed and inserted into a wearer's ear canal 22. Referring also to FIGS. 2-10, the earplug 20 also preferably includes an outer skin 28 that provides sufficient longitudinal rigidity to facilitate insertion the ear canal 22. The earplug of this invention may further include decorative and/or promotional markings 27 (FIGS. 8-10), such as trademarks, logos, brand names, decorative artwork, printed indicia, advertising, and the like, disposed thereon.

The earplugs of the present invention advantageously generate less manufacturing waste than prior art foam casting and die cutting processes. The present invention is also simpler to manufacture and more comfortable to the user than prior art composite ear plugs. Moreover, these extruded earplugs provide the unexpected benefit of greater sound attenuation than earplugs of the prior art. Further, the decorative and/or promotional markings may provide for an esthetically pleasing appearance. Further still, the markings may provide for brand and/or product differentiation and thereby create economic value.

Referring now to FIGS. 2-7, the body 24 preferably has a consistent transverse dimension (e.g., cross-sectional diameter) B. The body 24 is preferably made from a compressible or deformable material such as a low-density thermoplastic polymer material. The material may be a solid or a cellular material. The material desirably has a slow recovery from compression or deformation. Particularly preferred are materials that recover substantially all of their initial volume at a relatively slow rate of recovery. For example, such suitable materials preferably have a rate of recovery sufficient to recover 90 percent or less of their initial volume within 40-45 seconds. These materials should also recover about 90 percent or more of their initial volume within about 90 seconds. These parameters are set forth in greater detail in NTP 67 SAF Compression Test and NTP 102 SAF Recovery Test, published in 1996 by Norton Performance Plastics Corporation, and which are fully incorporated by reference herein. In addition to the foregoing rate of recovery parameters, in order that the earplug 20 can conform to the shape of a wearer's ear canal 22 (FIG. 1), it is preferable that the body material provides a structure that is compressible by hand to down to about 40 to 50% or less of its original transverse dimension (e.g., diameter in the embodiment shown).

Preferred materials for forming the body 24 are low-density thermoplastic materials such as PVC. Alternate materials that may be useful include thermoplastic elastomers such as thermoplastic polyolefins/ethylene-propylene (PEP OR EPDM), thermoplastic block copolymers/styrene-butadiene (SBS) and styrene-isoprene (SIS), thermoplastic polyester, thermoplastic polyurethane (PU)/polyester/polyether, thermoplastic vulcanizates, melt processible rubbers, polyamide blocks, thermoplastic rubber, and viscoelastic polyurethane. Santoprene(V thermoplastic rubber sold by Advanced Elastomer Systems may also be used. The material selected has a density sufficient to produce earplugs having densities ranging from about 2 to 20 pcf (32 to 320 kg/m³), or more preferably, within a range of from about 6 to 12 pcf (96 to 192 kg/m³).

When the body 24 is formed from a cellular material, the cells may be formed either by mechanical incorporation of gases such as air, nitrogen or carbon dioxide into the base polymer under pressure or by the incorporation of a chemical blowing agent, such as one generating CO₂, into the polymer material. The chemical blowing agent is then activated, usually by heat, to form a cellular polymer material. The material may in either case be an open or closed cell material.

In a preferred embodiment, the primary ingredients used to fabricate the earplugs 20, 20′, 20″, 20′″, 20″″ (FIGS. 2-10) of the present invention, i.e., PVC and polymeric plasticizer, are used in substantially similar proportions as used to fabricate conventional die cut earplugs, so as to provide similar degrees of softness and slow, controlled recovery from compression. These properties are important for comfort and ease of insertion in the ear, i.e., to give the user enough time to insert the product into the ear in a compressed state and allow it to expand to create a contoured seal within the ear canal. A useful formulation for fabrication of earplugs 20, 20′, 20″, 20′″, 20″″ of the present invention is as follows:

1. 100 parts by weight of PVC resin. The resin should be of low or medium molecular weight (i.e., within a range of 0.55 to 1.1 inherent viscosity (ASTM D1243), with low molecular weight, i.e., 0.85 or less, preferred), because this tends to give a more uniform cell-structure. The particle size and porosity of the resin should be in a range suitable for extrusion of plasticized product (as opposed to plastisol processing or rigid PVC products). A preferred material is OXY 200™, supplied by Oxy Vinyls, L.P. of Dallas, Tex.

2. 60 to 140 parts plasticizer (ADMEX 523™ is one suitable example) to provide the slow recovery from compression. Other plasticizers, such as epoxidized soybean oil, can be blended with the ADMEX 523™ Available from Hüls America of Piscataway, N.J. to increase recovery rate if use of the ADMEX 523™ alone provides a recovery that is too slow.

3. Acrylic processing aid. This material provides melt strength, i.e., it allows the melt to be drawn down to thin membranes during the expansion. A useable range would be about 5 parts to 30 parts.

4. Nucleator. This is particulate material that helps to control cell size. Many types are usable and are effective at 0.1 part up to 20 parts, depending on the type.

5. Stabilizer. This material prevents decomposition of the PVC resin that otherwise may be problematic during processing at elevated temperature. A suitable range is about 0.5 to 10.0 parts.

6. External lubricant. (Optional) This can be a fatty acid, metallic soap, or a wax and its purpose is to prevent the melt from sticking to the hot surfaces inside the extruder. A suitable range is 0 to 5 parts by weight.

7. PVC Dispersion Resin. (Optional) This is to improve powder flow properties without affecting nucleation. A suitable range is 0 to 1 part by weight.

The cross-sectional shape of the body 24 is preferably circular (as shown in FIGS. 2-10), though it may also be otherwise shaped, such as polygonally or irregularly shaped. The preferred transverse dimension (diameter) B of the body is between about 5 mm and 20 mm, preferably between about 10 mm and 20 mm.

As mentioned hereinabove, the body 24 preferably includes a continuous skin 28 about its outer surface. This is particularly desirable where the body 14 is formed from a cellular material because the continuous skin 28 protects any open cells along the outer surface from harboring soil. The continuous skin 28 is preferably integrally formed as a result of the extrusion process. However, it can also be provided through chemical or mechanical treatment of the monolithic structure, or it can be provided as an additional layer.

The earplugs of this invention may further include decorative or promotional markings 27 disposed thereon, as shown in FIGS. 8-10. Markings 27 may include any that may be disposed on the surface of the earplug 20′″, 20″″, such as trademarks, logos, brand names, decorative artwork, color patterns, printed indicia, advertising, and the like, and combinations thereof. In a preferred embodiment, markings 27 are disposed on continuous skin 28 by a conventional process such as ink jet (or bubble jet) printing, laser jet printing, screen-printing, offset printing, dye extrusion, or stamping. Markings 27 may also penetrate the earplug 20′″, 20″″ or may even be diffused therein, such as by a dye extrusion process performed prior to the formation of skin 28. The fabrication of earplugs 20′″, 20″″ including markings 27 is discussed in further detail hereinbelow.

Markings 27 may be disposed on substantially any portion of the earplug 20′″, 20″″. Preferably markings 27 are disposed on a portion of the earplug 20′″, 20″″ not inserted into the ear of a wearer. For example, markings 27 may be disposed on end 26′″ as shown in FIGS. 8-10. Markings 27 may also be disposed along the periphery of body 24, which is herein referred to as the longitudinal surface (or may be referred to as a cylindrical surface for earplugs having a cylindrical shape), as shown in FIGS. 8 and 10. The artisan of ordinary skill will readily recognize that the longitudinal surface of earplugs having non-circular (e.g., polygonal) transverse cross-sections may also include markings 27 disposed thereon. Further, it is typically required to use inks and/or dyes that are known to be non-toxic, and preferably non-irritants. Examples of such inks and/or dyes may include conventional child-safe acrylic paints. Conventional food colorings may also be used, although such colorings tend to be water-soluble and may tend to bleed or smear with repeated use. Preferable inks and/or dyes are thus color fast, to nominally prevent bleeding, smearing or other degradation of markings 27.

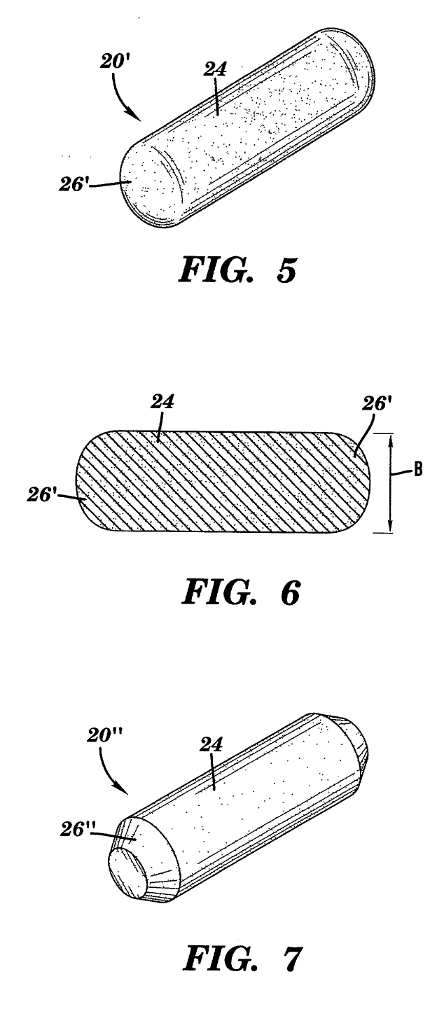

As shown, for example, in FIGS. 2, 5 and 7, the end portions 26, 26′, 26″ of the earplugs 20, 20′, 20″ may have any of a variety of configurations. In particular, it may be desirable to provide earplugs 20 having end portions 26 which are substantially flat or squared-off, such as in FIG. 2. Alternatively, in a preferred embodiment, generally convex end portions 26′ may be provided, as shown in FIGS. 5 and 6. Such a convex configuration may be advantageously attained without any extra operations by using a cutting operation to cut the extrudate substantially immediately upon emerging from the extruder die, as is discussed in greater detail hereinbelow. Additional geometries, such as the frusto-conical or tapered end portions 26″ shown in FIG. 7, may be provided using additional shaping or cutting steps known to those skilled in the art. The convex or tapered end portions 26′, 26″ generally facilitate insertion thereof into a wearer's ear canals.

The earplugs may include two end portions 26, 26′, 26″, which are of the same shape. Combinations may also be used, such as a conical end portion on one end 26″ of the earplug and a convex end portion on the other end 26′ of the earplug, so that the wearer may select which shaped end portion is more comfortable in his ear and insert that shaped end portion into his ear. In another embodiment, one end may be convex or conical, while the other end 26′″ (FIGS. 8-10) is substantially squared off and includes markings 27 disposed thereon.

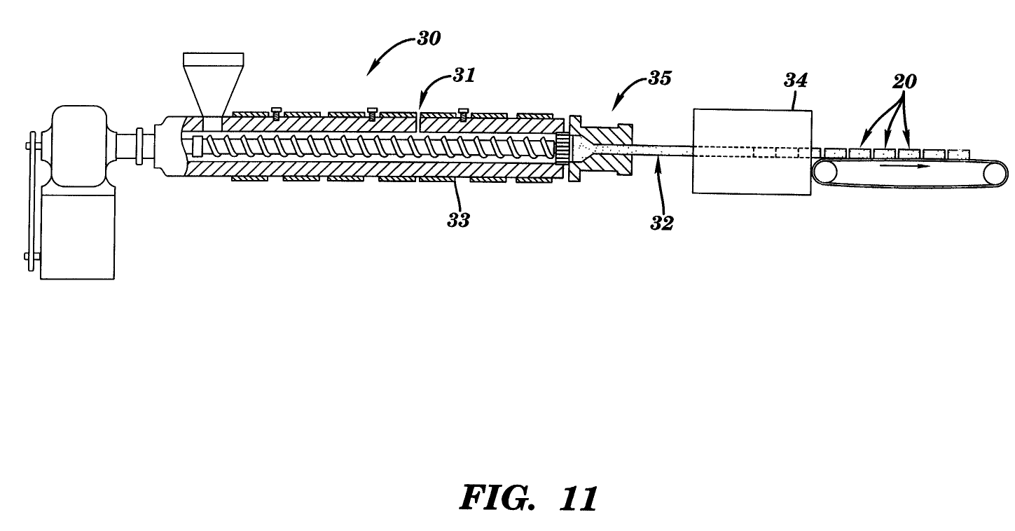

The manufacture of the earplugs may be performed with an apparatus as shown in FIG. 11. The body 24 is extruded from an extruder 30 to form a monolithic structure 32. Extruder 30 is substantially similar to those commonly used for making plasticized PVC products, with the exception that an injection nozzle 31 is disposed in the extruder barrel 33 so that the blowing agent may be added to the melted material. An extruder of this type is described in a technical correspondence bulletin published by the Monsanto Company entitled “Extrusion Foaming Technology for Santoprene Thermoplastic Rubber”, bulletin number TCD04287. The blowing agent may be a gas or liquefied gas such as CO₂. Other gases known to those skilled in the art also may be used. Through the action of heat and pressure, the blowing agent dissolves in the melted material. Upon emerging from the die 35, the release of pressure causes the blowing agent to vaporize or otherwise expand to form the cells of the foam. The monolithic extrudate 32 may then be cut and solidified, with the solidification means being dependent on the material used to form the extrudate. In a preferred embodiment, in which a PVC material is used, the extrudate is cooled in ambient air or by a temperature controlled air bath. Alternately, however, extrudates may be quenched by way of other traditional cooling methods, such as by treating with a liquid or gas bath to cool and solidify the extrudate.

After the monolithic structure 32 emerges from the extruder 30, it is fed to a cutting device 34 (shown schematically) which cuts the extrudate into discrete pieces of the desired length, preferably about 10 to 35 mm, thereby forming individual earplugs. Particularly preferred are earplugs having a length of about 17 to 25 mm. The monolithic structure may be cut into discrete pieces using any of a variety of conventional cutting devices such as a knife blade, hot wire, water jet, or laser, for example. In a preferred embodiment, the extrudate is cut to length in-line using a length cutter (i.e., rotary knife cutter) of the type commonly used in the plastic extrusion industry. Other suitable cutting machines include conventional high-speed wire and tubing cutters.

As mentioned hereinabove, in a preferred embodiment, it is desired to form earplugs having convex or otherwise shaped end portions. Such convex end portions 26′ may be advantageously formed without any additional process steps by cutting the extrudate at the die face of the extruder as the material emerges therefrom, prior to expansion and cooling thereof. After cutting, the end portions expand and cool, inherently forming a bowed, convex geometry. In addition, a film or skin is formed over the end surfaces as the foam material continues to expand and cool. The earplug thus produced has convex end portions 26′ and a film or skin 28 that extends over its entire exterior surface, including both the body 24 and end portions 26′. The skilled artisan will recognize that the exterior surface of body 24 (i.e., the cylindrical surface as shown) of the extrudate will be skinned over as an inherent function of the extruding process. Advantageously, the convex end portions 26′ tend to facilitate earplug insertion. Also, this embodiment has been shown to provide better sound attenuation than earplugs having unskinned end surfaces, and is generally less prone to harbor dirt or bacteria. This resistance to dirt also tends to improve the reusability and comfort of the earplugs. In the alternative, however, rather than cutting the extrudate prior to expansion and cooling, the extrudate may be cut after the expansion and cooling. This will provide an earplug having squared off ends 26, such as shown and described with respect to FIGS. 2-4 and 8-10.

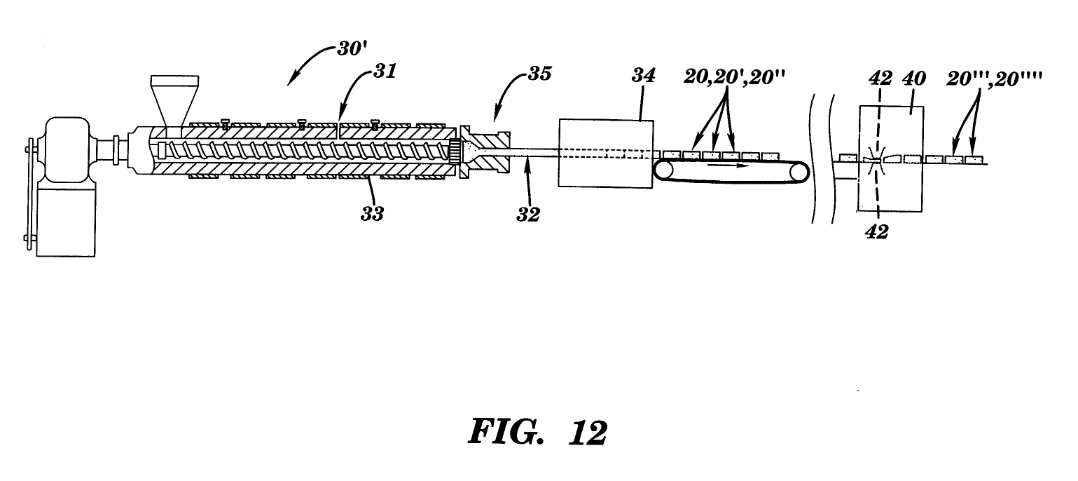

Earplugs including markings 27 may also be manufactured in a manner similar to that described above. For example, earplugs 20′″, 20″″ may be manufactured using extruder 30′ shown in FIG. 12. Extruder 30′ is substantially identical to extruder 30 except that it also includes a marking module 40. The earplugs are typically fed into marking module 40 after sufficient cooling. Marking module 40 includes at least one marking element 42. Marking element 42 may be any that is suitable for marking the extruded earplugs of this invention, such as but not limited to a stamp (e.g., a rubber stamp), an offset printing press, an ink jet (or bubble jet) print head, a laser print head, a dye jet, and the like.

In one embodiment, earplugs are fed into marking module 40 wherein they are stamped (e.g., pressed) with markings 27. For markings 27 in which high resolution is not required, a simple rubber stamp may provide an economical method of marking. When higher resolution is required, such as in detailed artwork or indicia, an offset press may be preferred. The stamped or pressed, (and generally flattened) earplugs emerge from marking module 40, after which they slowly recover substantially all of their initial volume. It is generally important that the stamping process involve sufficient pressure to adequately flatten at least a portion of the earplugs 20′″, 20″″ and impress the markings 27 into a surface thereof, but not so much pressure so as to damage (e.g., by cutting) the surface or skin thereon.

In another embodiment, earplugs are fed into marking module 40 wherein they are pressed relatively flat and further fed in close proximity to an ink jet (or bubble jet) print head, which disposes the desired markings thereon. As described above, the flattened earplugs, including markings 27 on a surface thereof, emerge from marking module 40, after which they slowly recover substantially all of their initial volume. The slow recovery rate from compression of the materials used to form the earplugs renders them well suited for the use of an ink jet (or bubble jet), or laser marking element 42.

Moreover, while a process in which the earplugs 20′″, 20″″ are flattened prior to marking has been described, the artisan of ordinary skill will readily recognize that it is not necessary to flatten the earplugs when using an ink jet (or bubble jet), or laser marking element 42. For example, earplugs 20′″, 20″″ may be fed past an ink jet (or bubble jet), or laser print head, which marks the surface thereof.

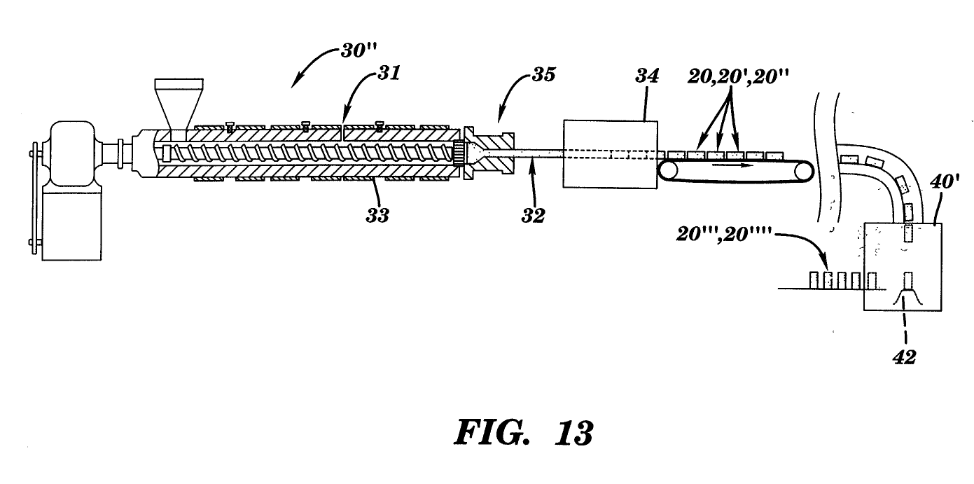

Referring to FIG. 13, an alternate embodiment of an extruder 30″ used to manufacture marked earplugs is shown. Extruder 30″ is substantially identical to extruder 30′, except that the earplugs are fed into marking module 40′ so that the ends 26, 26′, etc., thereof are generally superposed with marking element 42. Such orientation tends to facilitate marking the ends 26, 26′. In a further alternate embodiment, not shown in the figures, an extruder may include both marking modules 40, 40′, to mark both the cylindrical surface and an end portion of the earplugs.

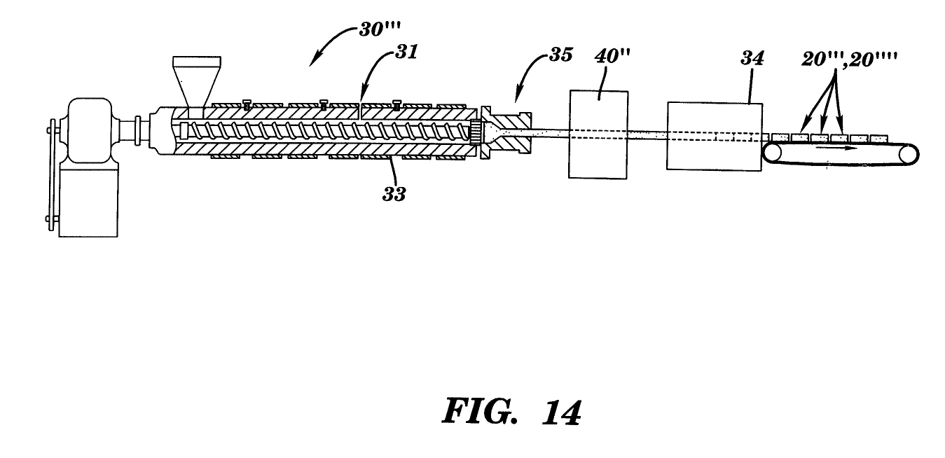

Referring now to FIG. 14, in yet another embodiment, extruder 30′″ may include a marking module 40″ between die 25 and cutting device 24. In one exemplary embodiment, marking module 40″ includes a dye-jet, including nozzles (not shown) for applying dye to the cylindrical surface of the extrudate. A dye-jet process may be useful for providing coding information (e.g., color coded lines such as those used for coding the resistance value of electrical resistors) to indicate the size, sound attenuation, or other characteristics of the earplugs, or for providing an artistic “striped” appearance (as in a candy cane).

Irrespective of the markings 27, extruded earplugs 20, 20′, 20″, 20′″, 20″″ of this invention may be made in substantially any color through the use of pigments, although their natural color is typically bright white. The bright white color is generally favorable both because it connotes cleanliness and because markings thereon may be readily seen.

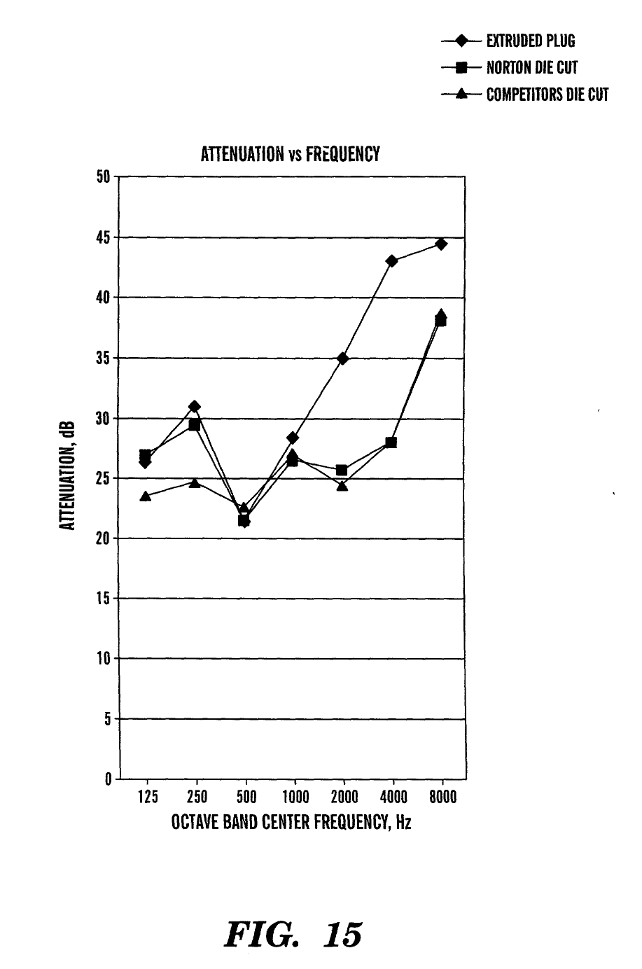

Turning now to FIG. 15, earplugs of the present invention have been tested for sound attenuation relative to conventional die cut earplugs. The (prior art) die cut plugs included the NORTON® SAFG™ Earplug manufactured by Norton Company of Worcester, Mass., and the EAR™ CLASSIC™ manufactured by E-A-R Specialty Composites Corporation of Indianapolis, Ind. As shown, the extruded earplugs of the present invention surprisingly provided substantially improved sound attenuation, especially within the critical 2000 to 8000 Hz range, i.e., the range of frequencies most likely to cause hearing damage.

Other properties, configurations, and advantages, of similar earplugs (not including markings 27) are disclosed in the '678 application. Example earplugs are also disclosed in the '678 application.

The modifications to the various aspects of the present invention described hereinabove are merely exemplary. It is understood that other modifications to the illustrative embodiments will readily occur to persons with ordinary skill in the art. All such modifications and variations are deemed to be within the scope and spirit of the present invention as defined by the accompanying claims.

What is claimed is:

1. A method of fabricating an earplug, said method comprising:
extruding a monolithic body of foamed elastomeric thermoplastic material about 5 to 20 millimeters in transverse dimension;
at least partially cutting the body into discrete pieces about 10-35 millimeters in length to form individual earplugs; and
disposing markings on a surface of the earplug.

2. The method of claim 1, wherein said markings are selected from the group consisting of decorative artwork, promotional markings, brand names, logos, indicia, trademarks, advertising, and combinations thereof.

3. The method of claim 1, wherein said disposing comprises a process selected from the group consisting of ink jet printing, bubble jet printing, laser printing, screen printing, offset printing, stamping, dye jet extrusion, and combinations thereof.

4. The method of claim 1, wherein said disposing includes offset printing.

5. The method of claim 1, wherein said markings are disposed on a skin which forms an outer surface of the monolithic body.

6. The method of claim 1, wherein said cutting is performed after said disposing.

7. The method of claim 1, wherein said disposing is performed after said cutting.

8. The method of claim 1, wherein said extruding comprises extruding a monolithic body of foamed elastomeric thermoplastic material about 10 to 20 millimeters in transverse dimension.

9. The method of claim 1, wherein said cutting comprises cutting the body proximate the die face as it emerges therefrom, prior to substantial expansion and cooling thereof, wherein a protective skin is formed at the ends of the pieces.

10. The method of claim 1, wherein said cutting further comprises forming convex end surfaces by cutting the extrudate as it emerges therefrom, prior to substantially complete cooling and expansion, wherein a convex, skinned surface is formed at the ends of the pieces as the extrudate expands and cools.

11. The method of claim 1, wherein the monolithic body has a density within a range of about 2 to 20 pcf (32 to 320 kg/m³).

12. The method of claim 10, wherein the monolithic body has a density of about 6 to 12 pcf (96 to 192 kg/m³).

13. The method of claim 1, wherein said monolithic body has a rate of recovery from compression to about 20 percent of its initial volume, and from compression under a 5 pound weight for 6 seconds, sufficient to recover about 90 percent or less of its initial volume in 45 seconds.

14. The method of claim 1, wherein said step of using an extruder further comprises extruding an extrudable material having a rate of recovery from being compressed under a 5 pound weight for 6 seconds, sufficient to recover about 90 percent or more of its initial volume in 90 seconds.

15. The method of claim 1, wherein the elastomeric thermoplastic material comprises:
95-105 parts by weight PVC resin;
60 to 140 parts by weight plasticizer;
5 to 30 parts by weight acrylic processing aid;
0.1 to 20 parts by weight nucleator;
0.5 to 10 parts by weight stabilizer;
0 to 5 parts by weight external lubricant; and.
0 to 1 part by weight dry flow promoter.

16. The method of claim 14, wherein the thermoplastic elastomeric material comprises:
100 parts by weight PVC resin;
80 parts by weight plasticizer;
7 parts by weight epoxidized soybean oil;
11 parts by weight acrylic processing aid;
6 parts by weight nucleator;
2.5 parts by weight stabilizer; and
0.5 parts by weight external lubricant.

17. The method of claim 1, wherein said method is a substantially continuous process.

18. A monolithic earplug formed by the method of claim 1.

19. An earplug sized and shaped for being received in the human ear canal, said earplug comprising:
an extruded monolithic body of foamed elastomeric thermoplastic material about 5 to 20 millimeters in transverse dimension having a length of about 10-35 millimeters; and
markings disposed on at least one surface of said earplug.

20. The earplug of claim 19, wherein said markings are selected from the group consisting of decorative artwork, promotional markings, brand names, logos, indicia, trademarks, advertising, and combinations thereof.

21. The earplug of claim 19, wherein said markings are disposed on an end portion thereof.

22. The earplug of claim 19, wherein said markings are disposed on a longitudinal surface of said monolithic body.

23. The earplug of claim 19, wherein said markings consists of non-toxic materials.

24. The earplug of claim 19, wherein said transverse dimension is in the range from about 10 to about 20 millimeters.

25. The earplug of claim 19, wherein said body has a substantially cylindrical outer surface along at least a majority of its length.

26. The earplug of claim 24, wherein said body has a substantially consistent transverse dimension throughout its length.

27. The earplug of claim 19, having a density in the range of between about 2 to about 20 pcf (32 to 320 kg/m³).

28. The earplug of claim 19, having a density in the range of between about 6 to about 12 pcf (96 to 192 kg/m³).

29. The earplug of claim 19, further comprising a continuous skin about the outer surface and end portions of said monolithic body.

30. The earplug of claim 28, wherein said markings are disposed on said continuous skin.

31. The earplug of claim 28, wherein said continuous skin is formed by cutting the body proximate the die face as the body emerges therefrom, prior to substantial expansion and cooling thereof, said continuous skin being formed as said thermoplastic material continues to expand and cool.

32. The earplug of claim 28, wherein said end portions are substantially convex.

33. The earplug of claim 19, having a rate of recovery from 80 percent compression sufficient to recover about 90 percent or less of its initial volume in about 45 seconds.

34. The earplug of claim 32, having a rate of recovery wherein after being compressed under a 5 pound weight for 6 seconds, the earplug recovers about 90 percent or more of its initial volume in about 90 seconds.

35. A monolithic earplug formed by the process of:
disposing a PVC-based material within an extruder under heat and pressure;
incorporating a blowing agent into the material;
extruding the material in a longitudinal direction from a die into an ambient environment wherein the blowing agent foams the extrudate, the extrudate having a transverse cross-sectional dimension of about 10 to 20 millimeters;
cutting the extrudate at a substantially 90 degree angle to the longitudinal direction as the extrudate emerges from the die and prior to substantially complete cooling and expansion thereof, wherein a convex, skinned surface is formed at the cut ends as the extrudate expands and cools to form a monolithic earplug having a density of about 6 to 12 pcf (96 to 192 kg/m³), and a rate of recovery from 80 percent compression sufficient to recover about 90 percent or less of its initial volume in 45 seconds, and after being compressed under a 5 pound weight for 6 seconds, to recover about 90 percent or more of its initial volume in 90 seconds; and
disposing markings on at least one surface of the earplugs.
